(19) United States
(12) Patent Application Publication
(10) Pub. No.: US 2024/0324885 A1
(43) Pub. Date: Oct. 3, 2024

(54) METHOD FOR OPERATING A HEARING DEVICE SYSTEM

(71) Applicant: SIVANTOS PTE. LTD., SINGAPORE (SG)

(72) Inventor: JULIUS KRIEG, ERLANGEN (DE)

(21) Appl. No.: 18/619,689

(22) Filed: Mar. 28, 2024

(30) Foreign Application Priority Data
Mar. 28, 2023 (DE) 10 2023 202 804.2

(51) Int. Cl. A61B 5/01; G01K 1/20; G01K 13/20; H04R 25/00

(52) U.S. Cl. CPC A61B 5/01; G01K 1/20; G01K 13/20; H04R 25/00

(57) ABSTRACT

The invention relates to a method for operating a hearing device system which has two hearing devices, in particular hearing aids, each with a temperature sensor. A current temperature value is determined by means of each temperature sensor, and a discrepancy between the two temperature values is determined. A state of health of a user of the hearing device system is estimated with the aid of the discrepancy. The invention further relates to a hearing device system.

### CROSS-REFERENCE TO RELATED APPLICATION

This application claims the priority, under 35 U.S.C. § 119, of German Patent Application DE 10 2023 202 804.2, filed Mar. 28, 2023; the prior application is herewith incorporated by reference in its entirety.

### FIELD AND BACKGROUND OF THE INVENTION

The invention relates to a method for operating a hearing device system and to a hearing device system. The hearing device system has two hearing devices, each with a temperature sensor.

Persons who are suffering from a reduction in hearing ability conventionally use a hearing aid. Ambient sound is thereby converted into an electrical (audio/sound) signal, usually by means of a microphone, that is to say an electromechanical sound transducer, and an electrical signal is created. The electrical signals are processed by way of an amplifier circuit, and introduced into the person's auditory canal by means of a further electromechanical transducer in the form of a receiver. The amplifier circuit is a constituent part of a control unit of the hearing aid. The sound signals are usually also processed, a signal processor of the amplifier circuit conventionally being used for this purpose. The gain is matched to any hearing loss of the hearing device wearer.

The reduction in hearing ability is usually age-related. There are often additional syndroms for the person using the device because of their age, which may lead to a worsening of their state of health. It is also possible for accompanying diseases to occur because of the reduction in hearing ability, for example depression. There may therefore be a lack of motivation that keeps the person from using or correctly operating the hearing aid, so that the reduction in hearing ability is not compensated for, which may exacerbate the depression. Such depression usually sets in gradually, for which reason it may be recognized by the person themself as well as by other people only comparatively late. When the depression is recognized, it is usually highly advanced, so that a comparatively comprehensive treatment is required.

### SUMMARY OF THE INVENTION

It is accordingly an object of the invention to provide a method and system which overcome the above and other disadvantages of the heretofore-known devices and methods of this general type and which provides for a particularly suitable method for operating a hearing device system and a particularly suitable hearing device system in which, in particular, convenience and/or functionality are increased.

With the above and other objects in view there is provided, in accordance with the invention, a method of operating a hearing device system with two hearing devices, in particular hearing aids, each having a temperature sensor. The method comprises:
determining a current temperature value by way of each temperature sensor to acquire two temperature values;
determining a discrepancy between the two temperature values; and
estimating a state of health of a user of the hearing devices based on the discrepancy between the two temperature values.

In other words, the method is used to operate a hearing device system which has two hearing devices. The hearing devices are intended and adapted to be worn on the human body. In other words, in the intended state the hearing devices are worn by a wearer, who is also referred to as a user. Each hearing device preferentially comprises a holding apparatus, by means of which fastening on the human body is possible. One of the hearing devices is assigned in particular to a left ear of the wearer and the other to a right ear of the wearer, so that the system is a binaural hearing device system. For example, the two hearing devices are designed identically to one another. As an alternative thereto, they are different but at least similar. Expediently, the two hearing devices are configured mirror-symmetrically with respect to one another, at least externally, so that adaptation to the different ear shapes is available.

For example, the hearing devices are earphones or each comprise an earphone. These are configured for example as so-called in-ear, on-ear and over-ear earphones. For example, the earphones are used to reproduce information and/or music, and/or the earphones are used for noise suppression and are so-called noise-canceling earphones. Particularly preferentially, however, each hearing device is a hearing aid. The hearing aids are used to assist a person suffering from a reduction in hearing ability. In other words, each hearing aid is a medical device by means of which, for example, a partial hearing loss is compensated for. The respective hearing aid, by way of example, is a “receiver-in-the-canal” hearing aid (RIC; ex-receiver hearing aid), an “in-the-ear” hearing aid, an “in-the-canal” hearing aid (ITC) or a “completely-in-canal” hearing aid (CIC). Alternatively, the respective hearing aid is a “behind-the-ear” (BTE) hearing aid, which is worn behind the pinna. If the respective hearing device is a hearing aid, the respective hearing device is intended and adapted for example to be arranged behind the associated ear or inside an auditory canal of the respective ear. Expediently, one of the hearing devices is therefore intended and adapted to be worn on or in the right ear and the other hearing device is intended and adapted to be worn on or in the left ear.

Each hearing device preferably comprises a microphone, which is used to record sound. In particular, an ambient sound or at least a part thereof is recorded during operation by means of the respective microphone. The respective microphone is, in particular, an electromechanical sound transducer. The respective microphone has for example only a single microphone unit, or a plurality of microphone units which interact with one another. Each of the microphone units expediently has a membrane that is set in oscillation with the aid of sound waves, the oscillations being converted into an electrical signal by means of a corresponding pickup device, such as a magnet which is moved in a coil. By means of the respective microphone unit, it is therefore possible to create an audio signal that is based on the sound to which the microphone unit is exposed. The microphone units are, in particular, configured to be unidirectional. The microphone is expediently arranged at least partially inside a housing of the hearing device and is therefore at least partially protected.

Each hearing device further comprises a control unit, which is coupled to the respective microphone. The control unit expediently comprises a signal processing unit or is formed by the latter. The signal processing unit is used in particular to further process and/or at least analyze the audio signal(s) created by means of the microphone. In particular, the audio signal is processed by means of the control unit so that an output signal, which is modified in comparison with the audio signal, is created. In particular, particular frequencies of the audio signal are amplified by means of the control unit, adaptation to any hearing loss of the wearer preferably taking place. For example, the signal processing unit has a plurality of analog components. Expediently, the signal processing unit or at least the control unit comprises a digital sound processor (DSP). Expediently, the (sound) processor is configured to be programmable.

Preferably, each hearing device has a receiver, which is used in particular to emit the respective output signal. The respective output signal in this case is, in particular, an electrical signal. Expediently, the receiver is coupled with the control unit, and in particular connected thereto in terms of signal technology. Depending on the configuration of the hearing device, in the intended state the respective output apparatus is arranged at least partially inside an auditory canal of the wearer of the hearing device, that is to say a person, or is at least acoustically connected thereto.

In particular, each hearing device is wireless and is intended and adapted to be inserted at least partially into an auditory canal. Particularly preferentially, the respective hearing device comprises an energy storage unit by means of which an energy supply is provided. Preferably, each hearing device has a communication instrument, which in particular comprises a radio system. It is therefore possible to connect the two hearing devices to one another in terms of signal technology. During operation, for example, processing of the respective possible output signal takes place as a function of the audio signal created by the other respective hearing device.

Each hearing device further has a temperature sensor. The temperature sensor is used to record/measure a temperature and is suitable, in particular configured, for this purpose. The temperature sensors assigned to the two hearing devices are expediently designed identically to one another, so that they preferably have the same time-dependent behavior in the event of temperature changes.

Each temperature sensor is used in particular to record/measure a temperature in the region of the respective ear of the user or is at least suitable for this purpose. Expediently, in the intended worn state of each hearing device, the respective temperature sensor is located in the region of the eardrum of the respectively assigned ear. In the intended state, the temperature sensor is preferably located in the region of the respective eardrum and/or in the respective auditory canal or is at least oriented in this direction. As an alternative thereto, the temperature sensor is intended and adapted to record a temperature inside a housing of the respective hearing device. For example, the temperature sensor is also used here to determine an operating temperature of the possible hearing device.

For example, the respective temperature sensor is a temperature-dependent resistor, which in the intended (worn) state of the respective hearing device is applied in the region of the respective ear so that there is mechanical contact. As an alternative thereto, the temperature sensor is for example an infrared sensor by means of which contactless recording of a respective temperature is made possible.

According to the method, a current temperature value is determined by means of each temperature sensor. In particular, for this purpose a temperature is measured by means of the respective temperature sensor. For example, the temperature in the region of the respectively assigned ear/auditory canal is measured and the respective current temperature value is created therefrom. For example, the current temperature value, which in particular is also referred to only as the temperature value, is an analog value or expediently a digital value. Preferably, a respective current temperature value is therefore determined for each ear.

In a further working step, a discrepancy between the two temperature values is determined. For this purpose, for example, the respective temperature value is transmitted from one hearing device to the other hearing device. As an alternative thereto, the two (current) temperature values are exchanged between the two hearing devices and the respective discrepancy is determined in each of the hearing devices. Preferentially, the discrepancy is created with a sign. In order to determine the discrepancy, in particular the difference between the two temperature values is created and one is therefore subtracted from the other. This suitably always takes place in the same way, so that for example the temperature value assigned to the right ear is always subtracted from the temperature value assigned to the left ear. As an alternative thereto, in order to determine the discrepancy a check is merely made as to which temperature value is higher, so that the discrepancy is only determined qualitatively.

In a further working step, a state of health of the user of the hearing device is estimated with the aid of the discrepancy. In order to estimate the state of health, the absolute value of the temperature values is not, or at least not only, taken into account, but rather the discrepancy, in particular the difference, between them. In particular, calibration of the two temperature sensors is therefore not required, for which reason production costs are reduced.

In the presence of some diseases, the activity of the brain of the user is distributed less uniformly between the two lateral sides (hemispheres) of the brain than in the absence of such a disease. Because of the different activity of the hemispheres, the blood circulation is less symmetrical, for which reason there is a temperature difference between the two halves of the brain. With the aid of the discrepancy of the temperature values, a conclusion is therefore drawn relating to a different blood circulation in the brain, and the state of health is estimated therefrom. In the case of a substantially smaller discrepancy, it is assumed that there is no pathological change. Conversely, it is estimated that the state of health is worse when the discrepancy is greater.

For example, in order to estimate the state of health the likelihood of the presence of a disease, such as depression, in particular a depressive episode, is determined. For example, only a binary determination of the presence of the depression takes place in this case, or the likelihood of a severity of the disease, in particular the depression, is also determined. In other words, in particular a likelihood of the presence of mild depression, a likelihood of the presence of moderate depression and/or a likelihood of the presence of serious depression is determined. If, in particular, the likelihood of the presence of depression is increased, in particular a comparatively poor state of health is estimated. The likelihood in this case is in particular increased when the discrepancy is greater.

For example, depression, increased aggression, schizoid behavior, social withdrawal and/or a listless/affective state is used as such a deterioration of the state of health. With these, the blood circulation in the left half of the brain is reduced. In summary, in particular the likelihood of a deteriorated state of health is greater when the current temperature value assigned to the left ear is lower (cooler) in comparison with the temperature value assigned to the right ear.

With the aid of the method, a state of health of the user may therefore be derived or at least estimated, no additional action needing to be carried out by the user. Convenience for them and a functionality of the hearing device system are therefore increased. It is possible here in particular to recognize a possible depression, an increased aggressive behavior, a schizoid, unmotivated, listless or affective state of health and/or social withdrawal early on, preferably to estimate the presence thereof, so that this may subsequently be checked suitably by a medical professional and an appropriate diagnosis may be made. In other words, a medical professional or another person from the medical field is provided with an indication in respect of the possible presence of a corresponding disease, so that this may be checked suitably. Comparatively early recognition of such a disease is therefore possible, for which reason a treatment may already take place early on before the disease is comparatively severe or untreatable. The consequences may also be mitigated in this way if the respective disease is actually present.

For example, a setting of one of the hearing devices or at least of a hearing device system may be modified as a function of the estimated state of health. For example, a message may be issued in this case, preferably for the user or another person. For example, the setting is always modified after the estimation of the state of health or only if there is a change in the estimated state of health. For this purpose, in particular, after the estimation of the state of health, the latter is stored, and the method is carried out several times, for which reason the state of health is estimated several times and respectively stored. A change in the estimated state of health may therefore be established, the time profile of the estimated state of health being available.

Preferably, the two temperature values are exchanged between the two hearing devices, preferably by means of the possible communication instrument. The latter is based for example on a Bluetooth standard. Particularly preferentially, however, the hearing device system also comprises a portable device such as a smartphone, tablet or wearable, on which for example a so-called app or another program, by means of which the method is at least partially carried out, runs. The temperature values are expediently transmitted to the portable device, by means of which in particular the discrepancy is determined and the state of health is estimated. The requirements for the hearing devices are therefore reduced, for which reason production costs are reduced.

There is for example a time lag, which is for example stochastic or always the same, between the determination of the two temperature values. Particularly preferentially, however, the two temperature values are determined simultaneously, so that in particular a synchronous measurement of the respective temperatures takes place. Preferably, the temperature is therefore measured synchronously for the ears. The temperature that is assigned to the two ears is therefore determined in the same state of the user, for which reason the possible discrepancy is not caused by modified circumstances of the user. Expediently, therefore, a synchronization of the two hearing devices is initially carried out in the method, particularly at a restart of the hearing device system, so that a corresponding synchronization is required only once. As an alternative thereto, repeated synchronization takes place, for example every hour, so that a time lag resulting for example from manufacturing tolerances of the hearing devices is limited. In particular, a corresponding timestamp is stored in the respective temperature value when measuring the respective temperature, which is therefore configured in the manner of a vector/tuple. For each downstream evaluation, it is therefore also possible to determine the instant at which the respective temperature was determined, in particular measured.

For example, the respective temperature value is determined only once, and therefore so is the discrepancy with the aid of which the state of health is estimated. In other words, a corresponding state of health is respectively estimated with the aid of each discrepancy, so that in particular there is a comparatively detailed time profile of the estimated state of health if the method is carried out several times. Particularly preferentially, however, each temperature value is measured several times, the time intervals in between preferably being constant. In particular, the time interval in between is between 1 second and 10 seconds, and preferably substantially equal to one hour or 30 minutes. In other words, two corresponding current temperature values are determined every hour. With the aid of each pair of temperature values, which in particular are measured simultaneously, the discrepancy is respectively determined. In other words, the corresponding temperature value is determined at the time intervals for each ear and the discrepancy is determined therefrom, so that there are a number of discrepancies. A measure of central tendency of these discrepancies is determined, for example the mean value, such as the arithmetic mean or a weighted mean. As an alternative thereto, the median is used as the measure of central tendency.

The state of health is estimated with the aid of the measure of central tendency. In this way, the discrepancy between the two temperature values, which is induced because of external circumstances, does not lead to a vitiation of the estimated state of health. Such external circumstances may be that only one ear is exposed to direct sunlight and/or to the direct effects of an air-conditioner/heater. In other words, because of the measure of central tendency, statistical errors are not taken into account, or are taken into account to a smaller extent, so that an accuracy when estimating the state of health is improved. For example, a statistical property of the measure of central tendency, such as a variance or a standard deviation, is also ascertained. In particular, this is taken into account when estimating the state of health, so that the state of health is respectively estimated with the aid of each measure of central tendency.

For example, the state of health is determined directly by means of the measure of central tendency. As an alternative thereto, such a measure of central tendency is respectively created for a plurality of successive time windows. The state of health is in this case estimated with the aid of a further measure of central tendency of the measures of central tendency. For example, the mean value, such as an arithmetic or weighted mean, or the median is used as the further measure of central tendency. For example, the calculation rule for determining the further measure of central tendency is the same as the calculation rule for determining the measure of central tendency, or they are different. For example, the median may be used as the further measure of central tendency and the mean value may be used as the measure of central tendency.

Preferably, between 25 and 20 measures of central tendency, expediently 10 measures of central tendency, are used for creating each further measure of central tendency, so that 10 measures of central tendency are respectively determined per time window. In particular, all the time windows are constant and/or between 30 minutes and 5 weeks. Preferably, the length of each time window is less than or equal to one week. Particularly preferentially, one day is used as the time window. For each day that the user uses the hearing device system, a respective further measure of central tendency is therefore determined and the respective state of health is therefore estimated once. In other words, the state of health is respectively estimated once per day, or for each day. On the one hand, a workload is therefore reduced. On the other hand, a gradually deteriorating state of health can nevertheless be ascertained, for which reason the user may be informed of this comparatively early on.

For example, the state of health is estimated only with the aid of the further measure of central tendency. Expediently, a statistical property, such as the variance or a standard deviation, of the further measure of central tendency is determined. In particular, this is taken into account when estimating the state of health. For example, in the event of a comparatively large uncertainty that is contained in the statistical property, such as a large variance, it is assumed that there is no change or only a minor change in the state of health if the latter is also estimated with the aid of a previous state of health already estimated. In any event, at least a comparatively low accuracy in the estimation of the state of health is assumed. If the uncertainty is low, conversely, a comparatively high accuracy in the estimation of the state of health is assumed.

For example, each temperature value determined is used to estimate the state of health. Particularly preferentially, however, whether the user additionally touches one of the hearing devices during the determination of the temperature values is monitored. In other words, whether further contact takes place, for example with a hand or another extremity of the user, in addition to the desired application of the hearing device on the user is monitored. Such contact takes place, for example, inadvertently in order to adapt the position of the respective hearing device on the ear or in order to change a setting of the respective hearing device. If the user touches the corresponding hearing device, this temperature value is not taken into account when estimating the state of health. This is because at least a slight change in the temperature of the respective hearing device takes place because of the contact and may lead to vitiation of the discrepancy respectively determined. Such an influence on the estimation of the state of health is therefore excluded, for which reason the accuracy in the estimation is increased. In particular, the temperature values determined for a particular period of time after the touching of the respective hearing device by the user are also not taken into account, or the determination is paused. In this way, the state of health is therefore taken into account via the temperature values only of temperatures that correspond to the temperature prevailing in the respective ear. The recording of the contact takes place, for example, by evaluation of the audio signal created by means of the possible microphone and/or of a corresponding sensor, such as for example a proximity sensor or an acceleration sensor. In the event of a comparatively abrupt short-term change in the placement of the hearing device, for example, it is assumed that corresponding contact by the user has taken place.

For example, the state of health is estimated only with the aid of the discrepancy (or discrepancies) and for example with the aid of the possible statistical properties. Particularly preferably, however, further parameters are also taken into account when estimating the state of health. In particular, ambient parameters are taken into account here, expediently an ambient temperature. These are for example measured by means of a corresponding temperature sensor of one of the hearing devices, or expediently by means of a temperature sensor of the smartphone, if the latter is a constituent part of the hearing device system. Expediently, the ambient temperature is measured comparatively far away from the hearing devices, so that it is substantially independent of them. In other words, the measured ambient temperature is not influenced by the user, or at least is not influenced by the blood circulation in their brain. The ambient temperature is preferably measured/recorded simultaneously with the determination of the temperature values. If the ambient temperature is comparatively low but the discrepancy between the two temperature values is comparatively large, one of the ears is for example positioned at least partially underneath a hat, or the like. This may be deduced with the aid of the ambient temperature so that it does not lead to a vitiation when estimating the state of health, and an accuracy is therefore increased.

As an alternative thereto or in combination therewith, a user activity is additionally recorded when determining the temperature values, for which purpose a suitable sensor is expediently used. For example, a heart rate sensor or accelerometer is used in order to characterize sporting activity. The user activity is taken into account when determining the state of health. In particular, the nature of the user activity and/or the intensity of the user activity is recorded. In other words, the physiological state of the user is preferably recorded and taken into account when determining the state of health. In the case of a comparatively strenuous physical activity, for example, the formation of a comparatively large discrepancy is possible without this corresponding to a deteriorated state of health.

The hearing device system has two hearing devices, which in particular are configured as hearing aids. Expediently, one of them is intended and adapted to be arranged on or in a left ear of a user and the other on or in a right ear of the user. Each hearing device has a temperature sensor, the two temperature sensors being in particular identical to one another. In the intended state, each temperature sensor is located for example in the auditory canal, preferably in the region of an eardrum of the respective ear, or at least in the region of/next to a temporal lobe of the brain of the user, for example on the surface of the head at a position that corresponds to the region of a temporal lobe. Preferably, the hearing device system also comprises a portable device such as a smartphone, a tablet or a wearable, to which the hearing devices are connected in terms of signal technology, preferably by means of a radio link.

The hearing device system is operated by means of a method wherein a current temperature value is determined, preferably simultaneously, by means of each temperature sensor. A discrepancy between the two temperature values is determined, and a state of health of the user of the hearing device system is estimated with the aid of the discrepancy. For example, the estimated state of health is output by means of the possible portable device or at least stored therein. Preferably, the state of health is estimated by means of the portable device and/or the discrepancy is determined therein. In the method, the temperature values are initially transmitted by each hearing device to the portable device, by means of which the appropriate further processing takes place. The resources required in the hearing devices are therefore reduced.

It will be understood that the features, developments and advantages described in connection with the method may also be applied correspondingly to the hearing device system, and vice versa.

Other features which are considered as characteristic for the invention are set forth in the appended claims.

Although the invention is illustrated and described herein as embodied in a method for operating a hearing device system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a schematic view showing a wearer of a hearing device system, which comprises two hearing devices and a portable device;

FIG. 2 is a schematic diagram showing details of the hearing device system; according to the invention; and

FIG. 3 is a flowchart of a method for operating the hearing device system.

Parts that correspond to one another are provided with the same reference signs throughout the figures.

### DETAILED DESCRIPTION OF THE INVENTION

Referring now to the figures of the drawing in detail and first, in particular, to FIG. 1 thereof, there is shown a wearer 2 of a hearing device system 4 in a simplified fashion. The wearer 2, or user 2, is a human (person) who is suffering from a partial hearing loss. In order to remedy this, the wearer 2 wears two hearing devices 6 of the hearing device system 4. One of the hearing devices 6 is assigned to a left ear and the other hearing device 6 is assigned to a right ear of the user 2. The hearing device system 4 is therefore configured binaurally; it is a binaural hearing device system. The hearing device system 4 further has a portable device 8 in the form of a smartphone, which the wearer 2 likewise carries with them.

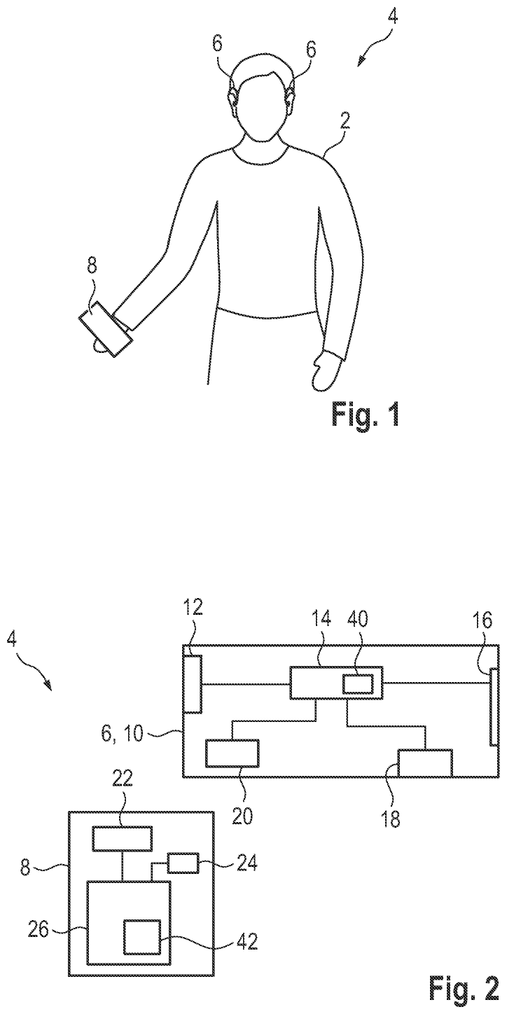

The hearing device system 4 is partially represented schematically in a simplified fashion in FIG. 2. It shows only one of the two hearing devices 6, which are configured mirror-symmetrically with respect to one another but otherwise identically. The exemplary hearing device 6 shown here is configured in the form of a hearing aid, which is intended and adapted to be inserted at least partially into the auditory canal of the respective ear of the wearer (user, hearing device wearer) 2. In other words, it is an “in-the-canal” hearing aid (ITC hearing aid). The hearing aid 6 comprises a housing 10, which is made of a plastic and is adapted to the shape of the respectively assigned auditory canal. A microphone 12 in the form of an electromechanical sound transducer, which is configured to be omnidirectional, is arranged inside the housing 10. The microphone 12 is coupled in terms of signal technology with a control unit 14, which comprises a signal processing unit with an amplifier circuit and a signal processor. The control unit 14 is further formed by means of circuit elements, such as for example electrical and/or electronic component parts. The signal processor is a digital signal processor (DSP) and is connected in terms of signal technology to the microphone 12 via an A/D converter.

A receiver 16 is coupled with, that is to say connected in terms of signal technology to, the control unit 14. By means of the receiver 16, which is an electromechanical sound transducer, an output signal provided by means of the control unit 14 is converted during operation into an output sound, that is to say into sound waves. During intended use, these are introduced into the auditory canal of the respective ear in the direction of the eardrum arranged therein.

A temperature sensor 18 is further arranged in the housing 10. The temperature sensor 18 is in one configuration a temperature-dependent resistor, which is embedded into an opening of the housing 10 and during intended use is applied on the skin of the auditory canal. It is therefore possible to measure the temperature of the skin of the user 2 in the region of the auditory canal by means of the temperature sensor 18. In an alternative configuration, the temperature sensor 18 is an infrared sensor which is oriented in the direction of the eardrum. By means of the temperature sensor 18, it is therefore possible to contactlessly measure the temperature of the eardrum, which substantially corresponds to the temperature of the temporal lobe of the brain of the user 2 assigned to the same side.

A communication apparatus 20, which is used to create a radio link with the portable device 8, is also arranged in the housing 10. The communication apparatus 20, and therefore also the radio link 22, comply in this case with a Bluetooth standard. Arranged in the housing 10, there is further an acceleration sensor by means of which it is possible to record an acceleration of the hearing device 6, which is caused for example because of a movement of the housing 10 in relation to the ear of the user 2 or because of a movement of the user 2 themself. The temperature sensor 18, the acceleration sensor and the communication apparatus 20 are controlled and powered by the control unit 14. By means of the latter, the microphone 12 and the receiver 16 are also supplied with electrical energy. The control unit 14 is powered by means of a battery that is also arranged in the housing 10.

The portable device 8, configured as a smartphone, has a further communication apparatus 22 which is connected via the radio link during operation to the communication apparatuses 20, 20 of the two hearing devices 6. The further communication apparatus 22 therefore complies with the Bluetooth standard. The portable device 8 comprises a further temperature sensor 24, by means of which an ambient temperature in the vicinity of the portable device 8 can be measured. The further communication apparatus 22 and the further temperature sensor 24 are connected in terms of signal technology to, and operated by means of, a further control unit 26 of the portable device 8.

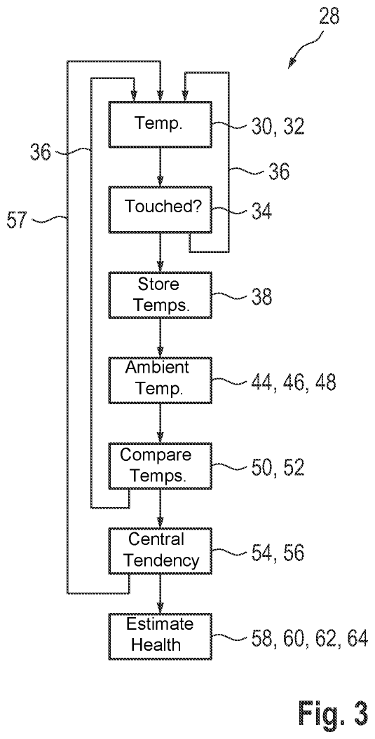

The control unit 14 and the further control unit 26 are intended and adapted to carry out a method 28, represented in FIG. 3, for operating the hearing device system 4. In other words, the hearing device system 4 is operated according to the method 28 and the method 28 is carried out at least partially by means of the respective control units 14, 26. In this case, in a first working step 30, a respective current temperature value 32, which is also referred to merely as a temperature value, is determined by means of the two temperature sensors 18. For this purpose, the temperature in the auditory canal is measured by means of each temperature sensor 18 and the measurement value as well as the instant at which the measurement of the respective temperature took place are stored in the respective temperature value 32. The respective instant in this case corresponds to the timestamp of the respective temperature value 32.

By means of the two hearing devices 6, the temperatures are measured simultaneously by the respective temperature sensor 18, so that the two temperature values 32 are determined at the same time. At a maximum, a time lag that may be established in particular with the aid of the timestamp, is less than 1 second. For this purpose, the two hearing devices 6 were synchronized before carrying out the method 28, so that clocks of the hearing devices 6 are operated substantially synchronously.

In a second working step 34, a check is made as to whether the user 2 has additionally touched one of the hearing devices 6 during the determination of the two temperature values 32 or whether such contact has taken place within 10 seconds before the determination of the temperature values 32. For this purpose, the audio signals created by means of the respective microphone 12 are evaluated in respect of noise that corresponds to an extremity of the user 2 touching the microphone 12, or at least the housing 10. The signals recorded by means of the acceleration sensor are also analyzed in respect of a comparatively abrupt and brief movement of the housing 10. In this case, it is likewise assumed that contact has taken place. If such contact has taken place, the temperature values 32 determined are discarded and the first working step 30 is carried out again after a time interval 36. The time interval 36 is in this case always 1 hour.

In summary, whether the user 2 additionally touches one of the hearing devices 6 during the determination of the temperature values 32 is therefore monitored, that is to say, whether further touching of the hearing device 6 by the user 2 takes place in addition to the touching of the respective ear. In this case, the temperature values 32 determined are not taken into account. Otherwise, a third working step 38 is carried out. In the latter, the respectively determined current temperature value 32 is initially stored by each hearing device 6 in a memory 40 of the respective control unit 14. In addition, the current temperature value 32 is transmitted from the control unit 14 by means of the communication apparatus 20 to the portable device 8 and is received there by means of the further communication device 22. The received temperature values 32 are saved in a further memory 42 of the further control unit 26. Should an error occur during the transmission of the temperature values 32, another exchange is possible since they are saved in the respective memory 40, 42.

In a fourth working step 44, an ambient temperature 46 is recorded by means of the further temperature sensor 24. A user activity 48 is also recorded in the fourth working step 44. For this purpose, the acceleration sensors of the two hearing devices 6 are interrogated by the portable device 8, specifically the further control unit 26, and an acceleration sensor (not represented in more detail) of the portable device 8 is read out. With the aid of the patterns contained in the measurement signals, a nature and an intensity of the user activity 48 being carried out during the determination of the temperature values 32 are ascertained. In summary, the ambient temperature 46 and the user activity 48 then taking place are therefore additionally recorded during the determination of the temperature values 32. These are stored in the further memory 42 while being provided with the respective timestamp.

In a subsequent fifth working step 50, a discrepancy 52 between the temperature values 32 is determined. For this purpose, the temperature value 32 that has been determined by means of the hearing device 6 assigned to the right ear of the user 2 is subtracted from the temperature value 32 determined by means of the hearing device 6 assigned to the left ear of the user 2. The discrepancy 52 determined is provided with the respective timestamp and is likewise saved in the further memory 42. After the time interval 36 has elapsed, that is to say after one hour, the first working step 32 is carried out again.

After the first working step 32 has been carried out ten times, that is to say after 10 hours, a sixth working step 54 is carried out. In the latter, a measure of central tendency 56 of the discrepancies 52 determined in the 10 preceding hours is determined. The measure of central tendency 56 corresponds here to the median of the discrepancies 52. The first working step 32 is subsequently carried out again.

After a time window 57, which lasts one week, a seventh working step 58 is carried out. In the latter, a further measure of central tendency 60 of the measures of central tendency 56 determined up to this instant is created. The arithmetic mean of the measures of central tendency 56 is in this case used as the further measure of central tendency 60. A statistical property 62, namely the variance, of the further measure of central tendency 60 is also determined.

With the aid of the further measure of central tendency 60, a state of health 64 of the user is estimated. Since the measures of central tendency 56 are used, each temperature value 32 is therefore measured several times and the discrepancy 52 is respectively determined, and the state of health 64 of the user 2 is estimated with the aid of the discrepancies 52. A corresponding measure of central tendency 56 is respectively created in this case for the plurality of successive time windows 57, the state of health 64 being estimated with the aid of the further measure of central tendency 60 that has been created with the aid of the measures of central tendency 56.

Specifically, if the temperature values 32 assigned to the left ear of the user 2 are significantly lower than the temperature values 32 assigned to the right ear of the user 2, which is ascertained with the aid of the further measure of central tendency 60, the likelihood that the left half of the brain of the user 2 is less well supplied with blood is increased. In this case, the further measure of central tendency 60 is negative. Since the measures of central tendency 56 are used, each temperature value 32 is therefore measured several times and the discrepancy 52 is respectively determined, and the state of health 64 of the user 2 is estimated with the aid of the discrepancies 52.

Reduced blood circulation in the left half of the brain corresponds to a reduced neural activity of the left half of the brain. This is at least usually also the case when the user 2 is suffering from depression or the latter is beginning, as well as for example in the case of schizoid behavior or other social withdrawal. The statistical property 62, namely the variance, is also taken into account when estimating the state of health 64. If this variance is comparatively large, the likelihood that there is a deterioration in the state of health 64, that is to say in particular there is an estimated depression or the like, is reduced. If there is a small variance, on the other hand, the likelihood of the existence is increased.

When estimating the state of health, the respective ambient temperature 46 is also taken into account. For instance, in the case of a comparatively low ambient temperature 46 and comparatively large discrepancies 52, a reduced likelihood that there is a disease, that is to say a deterioration of the state of health 64, is assumed. This is because, in the case of low ambient temperatures 46, it is possible that the user 2 is wearing a hat or the like, the latter being for example pulled only over one of the two ears.

Further, the user activity 48 determined is also taken into account when estimating the state of health 64. This is because, in the case of strenuous physical activity, there may be comparatively large discrepancies 52 without a deterioration of the state of health 64 existing. If there is strenuous physical activity as the user activity 48, there is therefore a lower weighting of the discrepancies 52 than determined.

If a deteriorated state of health 64 of the user 2 is estimated, a corresponding message is issued by means of the portable device 8, so that the user 2 may consult a medical specialist. The latter may then carry out an examination of the user 2 and make an appropriate diagnosis. In this way, it is easier for the medical specialist to diagnose an incipient depression, especially since there is an indication of its possible existence.

The invention is not restricted to the exemplary embodiment described above. Rather, other variants of the invention may also be derived therefrom by a person skilled in the art without departing from the subject matter of the invention. In particular, all individual features described in connection with the exemplary embodiment may also be combined with one another in a different way, without departing from the subject matter of the invention.

The following is a summary list of reference numerals and the corresponding structure used in the above description of the invention:
2 wearer, user
4 hearing device system
6 hearing device
8 portable device
10 housing
12 microphone
14 control unit
16 receiver
18 temperature sensor
20 communication apparatus
22 further communication apparatus
24 further temperature sensor
26 further control unit
28 method
30 first working step
32 current temperature value
34 second working step
36 time interval
38 third working step
40 memory
42 further memory
44 fourth working step
46 ambient temperature
48 user activity
50 fifth working step
52 discrepancy
54 sixth working step
56 measure of central tendency
57 time window
58 seventh working step
60 further measure of central tendency
62 statistical property
64 state of health

1. A method of operating a hearing device system, the method comprising:
providing two hearing devices each having a temperature sensor;
determining a current temperature value by way of each temperature sensor to acquire two temperature values;
determining a discrepancy between the two temperature values; and
estimating a state of health of a user of the hearing devices based on the discrepancy between the two temperature values.

2. The method according to claim 1, wherein the hearing devices are hearing aids.

3. The method according to claim 1, which comprises determining the two temperature values simultaneously.

4. The method according to claim 1, which comprises measuring each temperature value several times, determining several respective discrepancies, and estimating the state of health based on a measure of a central tendency of the discrepancies.

5. The method according to claim 4, which comprises creating one such measure of central tendency for a plurality of successive time windows, and estimating the state of health with the aid of a further measure of central tendency of the measures of central tendency.

6. The method according to claim 5, which comprises determining a statistical property of the further measure of central tendency and taking the statistical property into account when estimating the state of health.

7. The method according to claim 1, which comprises monitoring whether the user additionally touches one of the hearing devices during a determination of the temperature values, and disregarding those temperature values that were determined while the user touched one of the hearing devices.

8. The method according to claim 1, which further comprises recording an ambient temperature when determining the temperature values and taking the ambient temperature into account when estimating the state of health.

9. The method according to claim 1, which further comprises recording a user activity when determining the temperature values and taking the user activity into account when estimating the state of health.

10. A hearing device system, comprising two hearing devices, each having a temperature sensor, and being configured to be operated by a method according to claim 1.

11. The hearing device system according to claim 10, wherein said hearing devices are hearing aids.
